(19) United States
(12) Patent Application Publication
(10) Pub. No.: US 2011/0112210 A1
(43) Pub. Date: May 12, 2011

(54) PMMA PASTE

(75) Inventors: Sebastian Vogt, Erfurt (DE); Hubert Buechner, Nuernberg (DE)

(73) Assignee: HERAEUS MEDICAL GMBH, Wehrheim (DE)

(21) Appl. No.: 13/001,890

(22) PCT Filed: Jun. 13, 2009
(86) PCT No.: PCT/EP09/04272

§ 371 Date: Dec. 29, 2010

(30) Foreign Application Priority Data
Jun. 30, 2008 (DE) 10 2008 030 312.7

(51) Int. Cl. A61L 24/06

(52) U.S. Cl. 523/116

(57) ABSTRACT

A PMMA paste is made from a mixture of (A) methyl methacrylate having a methacrylic acid content of less than 1% and a water content of less than 1% and (B) at least one polymethyl methacrylate having a Tg of more than 65° C. and a molar mass of greater than or equal to 200,000 g/mol. The methyl methacrylate fraction is sufficient for the PMMA paste to be autosterile, the weight ratio of (B) to (A) preferably being 10-50% (B) to 50-90% (A). The PMMA paste can be used to produce paste-like one-component bone cements and two-component bone cements.

### CROSS-REFERENCE TO RELATED APPLICATIONS

This application is a Section 371 of International Application No. PCT/EP2009/004272, filed Jun. 13, 2009, which was published in the German language on Jan. 1, 2010, under International Publication No. WO 2010/000384 A2 and the disclosure of which is incorporated herein by reference.

### BACKGROUND OF THE INVENTION

The subject matter of the invention includes a PMMA paste (polymethyl methacrylate paste), its use, and paste-like single-component bone cements or two-component bone cements containing this paste.

PMMA bone cements have been known for decades and trace back to the basic work of Sir Charnley (J. Charnley, “Anchorage of the femoral head prosthesis of the shaft of the femur,” J. Bone Joint Surg., 42: 28-30 (1960)). The basic composition of the PMMA bone cements has in principle remained the same since then. PMMA bone cements comprise a liquid monomer component and a powder component. The monomer component contains in general the monomer methyl methacrylate and an activator dissolved in this monomer (N,N-dimethyl-p-toluidine). The powder component comprises one or more polymers produced on the basis of methyl methacrylate and comonomers, such as styrene and methyl acrylate, by polymerization, preferably suspension polymerization, a radiopaque material, and the initiator dibenzoyl peroxide. When the powder components are mixed with the monomer components, due to swelling of the polymers of the powder components in the methyl methacrylate, a plastically deformable paste is produced. Simultaneously, the activator N,N-dimethyl-p-toluidine reacts with the dibenzoyl peroxide, which breaks down with the formation of radicals. The formed radicals initiate the polymerization of the methyl methacrylate. With progressing polymerization of the methyl methacrylate, the viscosity of the cement paste increases until the paste solidifies and is thus cured.

Basic mechanical requirements of PMMA bone cements, such as 4-point flexural strength, flexural modulus, and compression strength, are described in ISO 5833. For the user of the PMMA bone cement the non-adhesiveness property of the bone cement is of significant importance. The term “non-adhesiveness” is defined in ISO 5833. In conventional PMMA bone cements non-adhesiveness indicates that the cement, after the mixing of the components, has reached the processing phase by swelling of the polymers contained in the cement powder in the monomer. In principle, a PMMA bone cement must be non-adhesive, so that the user can form and apply the cement. The PMMA bone cement may not adhere to gloves and to application aids, such as mixing systems, crucibles, or spatulas.

One disadvantage of the conventional PMMA bone cements for cement manufacturers is that both the powder components and also monomer components must each be produced and packaged in sterile conditions twice. This means at least four sterile packing means are required for one bone cement package.

Another disadvantage of the prior PMMA bone cements for the medical user consists in that the liquid monomer components must be mixed with the powder components directly before the cement application in a mixing system or in crucibles. Here, mixing errors can occur easily, which could negatively affect the cement quality. After mixing the monomer components with the powder components, depending on the cement type, a certain amount of time must elapse until the cement paste is non-adhesive and can be applied. Thereafter, the user has a more or less short processing time available in which total endoprostheses can be positioned or bone cavities can be filled as in kyphoplasty and vertebroplasty. During the processing time, the viscosity of the cement paste changes due to the increasing swelling of the polymer particles in the monomer and the progressing polymerization of the monomer. The relatively short processing time is a significant disadvantage of the prior bone cements. Especially disadvantageous are short processing times in kyphoplasty and vertebroplasty. A cement, in particular for vertebroplasty and kyphoplasty, would be desirable in which the viscosity of the cement paste remains essentially constant for a time period of several minutes during the cement application.

### BRIEF SUMMARY OF THE INVENTION

The object of the invention therefore consists in developing a PMMA paste, which can be used as a starting material for the production of paste-like PMMA bone cements, which can overcome the problems of the known PMMA bone cements.

The PMMA paste according to the invention is formed from a mixture of:
A. methyl methacrylate having a methacrylic acid content of less than 1% and a water content of less than 1%, and
B. at least one polymethyl methacrylate having a Tg (glass transition temperature) greater than 65° C. and a molar mass greater than or equal to 200,000 g/mol,
wherein the methyl methacrylate portion is sufficiently high that the PMMA paste is inherently sterile (autosterile).

The term “methyl methacrylate” is to be understood both as pure methyl methacrylate and also methyl methacrylate contaminated with low quantities of other monomers, such as ethyl methacrylate, propyl methacrylate, and styrene, as long as these impurities do not exceed a total content of 5%.

Preferred is a polymethyl methacrylate having a molar mass greater than 300,000 g/mol, and especially preferred having a molar mass greater than 500,000 g/mol.

Advantageously, the polymethyl methacrylate is soluble at least to 80 weight percent in the methyl methacrylate.

### DETAILED DESCRIPTION OF THE INVENTION

Surprisingly, it was found that pastes having a portion of methyl methacrylate of greater than or equal to 50 weight percent are inherently sterile. This means that methyl methacrylate has a killing effect on microbial cells in the PMMA paste. Accordingly, PMMA pastes are preferred in which a weight ratio of 10-50% polymethyl methacrylate to 50-90% methyl methacrylate exists.

The invention also relates to the use of the PMMA pastes described above for the production of paste-like single-component bone cements and two-component bone cements. For this purpose, the PMMA paste is mixed with organic and/or inorganic filler materials and with radical initiators and/or accelerators for the production of single-component and two-component bone cements.

Preferably, organic and inorganic filler materials, which do not swell in methyl methacrylate and have a methyl methacrylate absorption of less than 25%, are mixed with the PMMA paste.

In particular, the PMMA paste according to the invention is used for the production of an agent for fixing total endoprostheses and revision endoprostheses.

The use of the PMMA paste according to the invention is especially advantageous for the production of a self-curing filler material for vertebroplasty, kyphoplasty, and femur-neck augmentation.

The PMMA paste according to the invention can also be used for the production of local active-ingredient releasing systems. Thus, it is possible, for example, to form ball-shaped or bean-shaped implants with PMMA bone cement containing an antibiotic and produced from the PMMA paste according to the invention, wherein these implants can be used as local active-ingredient releasing systems.

Additional pharmaceutically active ingredients from the groups of antibiotics, hormones, growth factors, and antiphlogistics can be mixed with the PMMA paste according to the invention. As antibiotics, primarily aminoglycoside antibiotics, glycopeptide antibiotics, fluoroquinolone antibiotics, lincosamide antibiotics, and oxazolidinone antibiotics can be considered. Here, gentamicin, tobramycin, amikacin, teicoplanin, vancomycin, ramoplanin, dalbavancin, moxifloxacin, ciprofloxacin, lincosamine, clindamycin, and linezolid are preferred.

The invention will be explained by the following examples, without however limiting the invention. Unless otherwise indicated, parts and percentages given refer to the weight as in the rest of the description.

### Example 1

### Production of a PMMA Paste 1

500 ml methyl methacrylate (Fluka) was stirred together with 2.0 g Dowex 50WX2 and 2.0 g sodium sulfate in a 500 ml Erlenmeyer flask for 2 hours at room temperature and then the ion exchanger and the sodium sulfate were separated by filtration. The water content of the methyl methacrylate of 0.1% was determined by Karl-Fischer titration. The gas-chromatographically determined methacrylic acid content lay at 0.08%.

Then, 105 g polymethyl methacrylate (molar mass ca. 500,000 g/mol, Tg 100-106° C.) was dissolved in 195 g of the previously treated methyl methacrylate in a 500 ml two-neck flask with exclusion of air (nitrogen flow) at room temperature under careful stirring. A bubble-free, very viscous, colorless paste was produced.

For testing the inherent sterility of the paste, spore strips (Bacillus subtilis ATCC 6633) were inserted into the paste. After storage for 72 hours at room temperature, the spore strips were removed and tested for sterility by incubation. No growth resulted.

### Example 2

### Production of a PMMA Paste 2

Paste 2 was produced analogously to the PMMA paste 1, wherein, however, a PMMA with a molar mass of ca. 700,000 g/mol and a Tg of 100-106° C. was used. The PMMA concentration was set at 30%.

### Example 3

### Production of a PMMA Paste 3

Paste 3 was produced analogously to the PMMA paste 1, wherein, however, a PMMA with a molar mass of ca. 1,200,000 g/mol and a Tg of 100-105° C. was used. The PMMA concentration was set at 28%.

### Example 4

### Production of a Two-Component Paste Cement

### Paste A:

22.80 g of the PMMA paste 1 was kneaded together with 1.50 g 1-cyclohexyl-5-ethyl-barbituric acid, 3.15 g zirconium dioxide, and 5.00 g polymethyl methacrylate semi-soluble in MMA.

### Paste B:

22.80 g of the PMMA paste 1 was kneaded together with 125 mg Aliquat 336, 0.5 mg copper(II)-2-ethylhexanoate, 3.15 g zirconium dioxide, and 5.00 g polymethyl methacrylate semi-soluble in MMA (grain size <63 μm, MMA absorption ca. 10%).

30 g of the paste A was kneaded with 30 g of the paste B. The resulting cement paste was immediately non-adhesive and could be shaped over a time period of ca. 5 minutes. Thereafter, the curing of the cement occurred within 4 minutes.

Test bodies were produced with the cement paste for determining the 4-point flexural strength and the flexural modulus according to ISO 5833 and for testing the Dynstat impact strength. The testing for the 4-point flexural strength and the flexural modulus was carried out after storage of the test bodies at room temperature for 24 hours and also after storage of the test bodies in water at 37° C. for 24 and 48 hours.

### Test Results:

4-point flexural strength (air/24 hr/room temperature): 49.9±0.8 MPa

Flexural modulus (air/24 hr/room temperature): 1985±53 MPa

4-point flexural strength (water/24 hr/37° C.): 65.5±1.1 MPa

Flexural modulus (water/24 hr/37° C.): 2604±29 MPa

4-point flexural strength (water/48 hr/37° C.): 71.8±1.7 MPa

Flexural modulus (water/48 hr/37° C.): 2726±74 MPa

Dynstat impact strength (air/24 hr/room temperature): 3.47±0.35

Dynstat flexural strength (air/24 hr/room temperature): 72.71±2.33.

### Example 5

### Production of a Two-Component Paste Cement

### Paste A:

21.20 g of the PMMA paste 2 was kneaded together with 1.50 g 1-cyclohexyl-5-ethyl-barbituric acid, 3.15 g zirconium dioxide, and 6.60 g polymethyl methacrylate semi-soluble in MMA.

### Paste B:

21.20 g of the PMMA paste 1 was kneaded together with 125 mg Aliquat 336, 0.5 mg copper(II)-2-ethylhexanoate, 3.15 g zirconium dioxide, and 6.60 g polymethyl methacrylate semi-soluble in MMA (grain size <63 μm, MMA absorption ca. 10%).

30 g of the paste A was kneaded with 30 g of the paste B. The resulting cement paste was immediately non-adhesive and could be shaped over a time period of ca. 5 minutes. Thereafter, the curing of the cement occurred within 4 minutes.

Test bodies were produced with the cement paste for determining the 4-point flexural strength and the flexural modulus according to ISO 5833 and for testing the Dynstat impact strength. The testing of the 4-point flexural strength and the flexural modulus was carried out after storage of the test bodies at room temperature for 24 hours and also after storage of the test bodies in water at 37° C. for 24 and 48 hours.

### Test Results:

4-point flexural strength (air/24 hr/room temperature): 52.0±1.2 MPa

Flexural modulus (air/24 hr/room temperature): 2080±52 MPa

4-point flexural strength (water/24 hr/37° C.): 64.2±2.3 MPa

Flexural modulus (water/24 hr/37° C.): 2548±51 MPa

4-point flexural strength (water/48 hr/37° C.): 72.1±1.5 MPa

Flexural modulus (water/48 hr/37° C.): 2803±59 MPa

Dynstat impact strength (air/24 hr/room temperature): 4.11±0.29

Dynstat flexural strength (air/24 hr/room temperature): 81.23±1.77.

### Example 6

### Production of a Two-Component Paste Cement for Vertebroplasty/Kyphoplasty

### Paste A:

20.80 g of the PMMA paste 3 was kneaded together with 1.50 g 1-cyclohexyl-5-ethyl-barbituric acid and with 13.0 g zirconium dioxide.

### Paste B:

20.80 g of the PMMA paste 1 was kneaded together with 125 mg Aliquat 336, 2.0 mg copper(II)-2-ethylhexanoate, and 13.0 g zirconium dioxide.

30 g of the paste A was mixed with 30 g of the paste B with the help of a double-cartridge system with a mounted static mixer. The resulting cement paste was immediately non-adhesive and could be shaped over a time period of ca. 7 minutes. Thereafter, the curing of the cement occurred within ca. 6 minutes.

Test bodies were produced with the cement paste for determining the 4-point flexural strength and the flexural modulus according to ISO 5833 and for testing the Dynstat impact strength. Testing of the 4-point flexural strength and the flexural modulus was carried out after storage of the test bodies at room temperature in air and at 37° C. in water for 24 hours.

### Test Results:

4-point flexural strength (air/24 hr/room temperature): 49.8±1.4 MPa

Flexural modulus (air/24 hr/room temperature): 2271±112 MPa

4-point flexural strength (water/24 hr/37° C.): 62.0±3.8 MPa

Flexural modulus (water/24 hr/37° C.): 3001±40 MPa

Dynstat impact strength (air/24 hr/room temperature): 3.67±0.24

Dynstat flexural strength (air/24 hr/room temperature): 65.78±4.41.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-10. (canceled)

11. A PMMA paste made from a mixture comprising:
A methyl methacrylate having a methacrylic acid content of less than 1% and a water content of less than 1%, and
B at least one polymethyl methacrylate having a Tg greater than 65° C. and a molar mass greater than or equal to 200,000 g/mol, wherein the methyl methacrylate comprises a sufficiently high portion of the mixture that the PMMA paste is inherently sterile and the mixture has a weight ratio of 10-50% B to 50-90% A.

12. The PMMA paste according to claim 11, wherein the polymethyl methacrylate has a molar mass greater than 300,000 g/mol.

13. The PMMA paste according to claim 12, wherein the molar mass is greater than 500,000 g/mol.

14. The PMMA paste according to claim 11, wherein the polymethyl methacrylate B is soluble at least to 80 weight percent in the methyl methacrylate A.

15. A paste-like single-component bone cement containing a PMMA paste according to claim 11.

16. A paste-like two-component bone cement containing a PMMA paste according to claim 11.

17. An agent for fixing total endoprostheses and revision endoprostheses, the agent comprising a PMMA paste according to claim 11.

18. A self-curing filler material for vertebroplasty, kyphoplasty, and femur-neck augmentation, the material comprising a PMMA paste according to claim 11.

19. A local active-ingredient releasing system comprising a PMMA paste according to claim 11.

20. A method for production of paste-like single-component bone cements or two-component bone cements, the method comprising mixing a PMMA paste according to claim 11 with organic and/or inorganic filler materials and with radical initiators and/or accelerators.

21. The method according to claim 20, wherein the PMMA paste is mixed with organic and inorganic filler materials, which do not swell in methyl methacrylate and which have a methyl methacrylate absorption of less than 25%.
